(19) United States
(12) Patent Application Publication
(10) Pub. No.: US 2022/0008365 A1
(43) Pub. Date: Jan. 13, 2022

(54) Treating Intraocular Retinoblastoma with Inhibitors of Histone Modification

(71) Applicant: Vanderbilt University, Nashville, TN (US)

(72) Inventors: Anthony B. Daniels, Nashville, TN (US); Debra L. Friedman, Nashville, TN (US)

(21) Appl. No.: 17/294,339

(22) PCT Filed: Nov. 14, 2019
(86) PCT No.: PCT/US19/61562

§ 371 Date: May 14, 2021

Related U.S. Application Data
(60) Provisional application No. 62/767,434, filed on Nov. 14, 2018.

(51) Int. Cl. A61K 31/165; A61K 31/4406; A61K 31/4045; A61K 38/15

(52) U.S. Cl. CPC A61K 31/165; A61K 38/15; A61K 31/4045; A61K 31/4406

(57) ABSTRACT

Methods of treating a proliferative conditions of the eye, comprising administering to a patient a therapeutically effective amount of a composition that comprises a compound that inhibits the biological activity of a mammalian histone deacetylase (HDAC), or a pharmaceutically acceptable salt thereof; and a pharmaceutical carrier.

### GOVERNMENT SUPPORT

This invention was made with government support under 1K08EY027464-01 awarded by the National Institutes of Health. The government has certain rights in the invention.

### BACKGROUND OF THE INVENTION

Retinoblastoma (RB) is the most common primary intraocular tumor in children. Historically, the mainstay of eye-conserving therapy was intravenous (IV) chemotherapy (“chemoreduction”) with carboplatin/etoposide/vincristine (CEV), which has led to 99% patient survival rates in developed countries. However, systemic chemotherapy is associated with side effects such as neutropenia, hearing loss, reduced fertility, and need for blood transfusions, and there is concern that current regimens might lead to secondary acute myelogenous leukemias later in life. Eyes with advanced tumors (Reese-Ellsworth [R-E] group V and International Classification of Retinoblastoma [ICRB] group D and E) are rarely saved by IV chemoreduction, and often require enucleation (eye removal) to achieve local control. In particular, IV chemotherapy has had poor success in eliminating subretinal seeds or vitreous seeds, which is seen when more advanced tumors begin to shed tumor cell clusters into the vitreous cavity of the eye or the subretinal space.

The recent introduction of microcatheter-based endovascular intra-arterial chemotherapy (IAC), using melphalan-based regimens, has led to dramatic improvements in globe salvage rates for these advanced eyes. In addition, direct intravitreal injections of chemotherapy (again, predominantly melphalan) have allowed eyes with extensive vitreous seeds to be saved, whereas previously those eyes with vitreous seeds (ICRB Group D/E or R-E Group V) were rarely salvageable. In centers such as the one at Vanderbilt University that offer these therapies, IAC and intravitreal chemotherapy have now dramatically reduced the rate of enucleation for RB. Thus the introduction of IAC and intravitreal chemotherapy have dramatically improved rates of eye salvage for retinoblastoma. At the same time, systemic adverse effects such as neutropenia, blood transfusions, reduced fertility, and increased risk of secondary cancers can all be avoided by delivering treatment directly to the tumor.

While new techniques like IAC and intravitreal chemotherapy have dramatically improved globe salvage for eyes with advanced RB, the current melphalan-based regimens for both delivery routes have significant ocular, neuroretinal and retinal vascular toxicity, and therefore often the globe is preserved but at the expense of vision. Rates of complications associated with intra-arterial melphalan approach 40%, and include blinding side effects such as retinal vascular occlusions and ophthalmic artery sclerosis, vitreous and choroidal hemorrhage, optic nerve swelling, retinopathy, ocular muscle myositis, cranial nerve palsies, and phthisis bulbi (where the eye becomes shrunken and painful and needs to be removed). Intravitreal injections of melphalan are known to cause a pigmentary retinopathy and to led to decreased retinal responses to light. At currently-used doses, each successive injection leads to a measurable (and cumulative) decrease in electroretinography responses, which are an objective clinical measure of retinal function. This has been demonstrated in both human retinoblastoma patients treated with intravitreal melphalan as well as in animal models of intravitreal melphalan injection. A full treatment course of intravitreal melphalan often consists of multiple weekly injections, with the knowledge that each injection, though eradicating disease, is reducing vision in the eye(s).

Newer drugs might be able to achieve tumor control without the ocular toxicity and visual morbidity associated with currently-used drugs. However, thus far, there has not been a small animal model that allowed IAC drug delivery to test the safety and toxicity profiles of alternative drugs. Instead, new drug testing and dose ranging has had to occur in the clinic, titrating until toxicity is reached in human infants with RB. There has been general acceptance in the field that melphalan is toxic to the retina and retinal vasculature, and consensus that new alternative options are needed.

Thus, there is a long felt need for a method of successfully treating retinoblastoma patients without the toxicity concerns of presently available treatments.

The present inventors have developed the first small animal (rabbit) model of IAC drug delivery, as well as a rabbit model of diffuse vitreous seeds along with a quantitative method for assessing drug efficacy in vivo. The present inventors have previously demonstrated, using melphalan, that we can determine the pharmacokinetics of drugs delivered via IAC in the various tissues of the rabbit eye, and determined the time-course and tissue drug levels. The present inventors have likewise determined the pharmacokinetics of several drugs when injected via direct intravitreal injection in vivo in rabbits. The present inventors have demonstrated that toxicity associated with various drugs injected either directly into the vitreous, or endovascularly to the eye via IAC can be measured. The present inventors have developed a battery of tests for assessing ocular and systemic side effects and retinal physiology. This includes structural measures of eye health such as optical coherence tomography, fundus photography, and histopathology, as well as functional measures of eye physiology such as electroretinography, fluorescein angiography, and optical coherence tomography angiography. In addition, several blood parameters such as complete blood counts are tracked during and subsequent to treatment.

Histones, the proteins that hold together DNA strands, are modified through acetylation/deacetylation and methylation/demethylation as a way to control gene expression and therefore regulate cellular function. In fact, protein complexes controlling histone modification are among the most commonly mutated genes across all human cancers. In particular, histone deacetylase (HDAC) inhibitors are scientifically attractive, and largely unexplored, potential targets for the treatment of retinoblastoma.

A number of HDACs have been identified. In humans, HDAC proteins comprise a family of 18 members, which are separated into four classes based on size, cellular localization, number of catalytic active sites, and homology to yeast HDAC proteins. Class I includes HDAC1, HDAC2, HDAC3, and HDAC8. Class I HDACs are ubiquitously expressed, largely restricted to the nucleus and in the case of HDACs 1, 2 and 3, known to deacetylate histones. They share a highly conserved and homologous N-terminal catalytic domain. Class II consists of six HDAC proteins that are further divided into two subclasses. Class IIa includes HDAC4, HDAC5, HDAC7, and HDAC9, which each contain a single catalytic active site. Class IIa HDACs display more tissue specific distribution and can translocate between the nucleus and cytoplasm. These enzymes display weak inherent catalytic activity and require higher order protein complexes, often containing HDAC3, to become catalytically competent deacetylases. Class IIb includes HDAC6 and HDAC10, which each contain two active sites, although only HDAC6 has two catalytically competent active sites. The Class IIb HDACs include HDACs 6 and 10 with HDAC6 being predominantly localized to the cytoplasm while little is known about the localization of HDAC10. The Class IIb HDACs uniquely contain two independently active and substrate specific catalytic domains and it is the N-terminal domain of HDAC6 that is responsible for the deacetylation of α-tubulin. Aspects of the present invention can inhibit a targeted HDAC (or specific classes of HDACs). Other aspects of the present invention include the use of pan-inhibitors.

HDAC1 binds Rb pathway proteins, and HDAC9 is overexpressed in retinoblastoma, particularly in more aggressive tumors. Currently, a handful of HDAC inhibitors (HDACi) are Food and Drug Administration (FDA)-approved for the treatment of blood cancers, such as belinostat (a pan-HDAC inhibitor) being used for the treatment of peripheral T-cell lymphoma. However, generally they are not used for solid tumors. In addition, a limitation of current HDAC inhibitors is that, at currently used doses and current systemic administration routes, HDAC inhibitors have to be given over prolonged periods of time in order to gradually effect change on tumor gene expression. In addition, we know that for the treatment of retinoblastoma, systemically administered antineoplastic agents do not achieve very high intraocular drug concentrations. In contrast, intra-arterial and intravitreal drug delivery achieve much higher intraocular drug concentrations, and the present inventors have demonstrated this in a rabbit model.

### SUMMARY OF THE INVENTION

One aspect of the present invention is a localized, intraocular method of treating eye cancer.

One aspect of the present invention is a method of treating intraocular tumors.

One aspect of the present invention is a method of treating retinoblastoma.

One aspect the present invention is a method for the manufacture of a medicament for use in treating eye cancer, including retinoblastoma.

One aspect of the present invention is a pharmaceutical composition comprising at least one HDAC inhibitor, particularly a composition suitable for intravitreal or intra-arterial delivery.

One aspect of the present invention is a pharmaceutical composition comprising at least one HDAC inhibitor in a suitable container for intravitreal or intra-arterial delivery.

One aspect of the present invention pertains to the use of a HDACi compound of the present invention for use in a method of treatment of the human or animal body by therapy. In one aspect, that use is treatment of a cancer of the eye. In one aspect, that cancer is retinoblastoma.

One aspect of the present invention pertains to a compound of the present invention and its use in the manufacture of a medicament for the treatment of a retinoblastoma by intravitreal or intra-arterial delivery.

One aspect of the present invention pertains to a method of treatment, comprising administering to a subject in need of treatment a compound or pharmaceutical composition of the present invention an effective eye cancer treating amount.

One aspect of the present invention pertains to a method of (a) regulating (e.g., inhibiting) cell proliferation; (b) inhibiting cell cycle progression; (c) promoting apoptosis; (d) promoting necroptosis, (e) promoting senescence, or (f) promoting necrosis, (g) promoting autophagy, (h) promoting another form of cell death, or (i) a combination of one or more of these, in vitro or in vivo, comprising delivery of a compound or composition of the present invention to the eye.

One aspect of the present invention pertains to a method of administering a HDACi, as defined herein, to a subject, comprising administering to said subject a pharmaceutical composition (e.g., formulation), as described herein, by intravitreal or intra-arterial delivery.

One aspect of the present invention pertains to a kit (or kit-of-parts) comprising: (a) a compound or pharmaceutical composition (e.g., pre-formulation, formulation) as described herein, preferably provided in a suitable container for intravitreal or intra-arterial delivery and/or with suitable packaging; and (b) instructions for use, for example, written instructions on how to administer the formulation, etc.

One aspect of the present invention pertains to a kit (or kit-of-parts) comprising: (a) a pharmaceutical composition (e.g., pre-formulation) as described herein, preferably provided in a suitable container and/or with suitable packaging; and (b) instructions for use, for example, written instructions on how to prepare a suitable pharmaceutical formulation from the composition (e.g., pre-formulation), and how to subsequently administer the formulation, etc.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

FIG. 1 is photographs showing treatment of an eye injected with human WERI-Rb1 cells. The first column is a saline treated eyes, and the second column is belinostat treated eyes in accordance with an aspect of the present invention.

FIG. 2 is a graph showing efficacy of melphalan treated eyes and belinostat treated eyes in accordance with an aspect of the present invention. The melphalan treated eyes showed a 93% reduction in tumor cells and the belinostat treated eyes showed a 95% reduction in tumor cells.

FIG. 3 is a set of graphs that show measurements of (absence of) retinal functional loss and (absence of) toxicity with intravitreal belinostat toxicity, at both the clinically-effective dose, and even at twice that dose. A wave amplitude and B wave amplitude are measured.

FIG. 4 is a set of graphs that show a comparison of retinal function following intravitreal injections of belinostat (350 μg) to standard of care intravitreal melphalan. The downward trend indicates retinal functional loss. NS=trend not significant.

FIGS. 5A, 5B, and 5C show histopathology examples of toxicity of the present invention compared to melphalan. FIG. 5A shows no evidence of retinal degeneration with belinostat, a compound of the present invention. FIG. 5B is untreated. FIG. 5C shows evidence of retinal degeneration with standard of care melphalan. This figure shows that the retina of a rabbit eye treated with intravitreal belinostat (even at twice the clinically-effective dose) retains perfect retinal architecture and structure, compared to a rabbit eye treated with standard of care intravitreal melphalan, where the retina can be seen to be completely atrophied from toxicity.

FIGS. 6A and 6B demonstrate that certain of the specific HDACs are expressed across nearly all patient tumors, while other HDACs are expressed in none, or only variably, across RB tumors of different patients. This demonstrates that the present invention does not necessarily have to include the inhibition of an entire class of histone modifiers (i.e., not a pan-HDAC inhibitor, etc.), but can include drugs that target less than all HDACs.

### DESCRIPTION OF THE INVENTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

In some aspects, a structure of a compound can be represented by a formula:

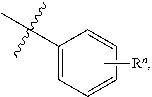

which is understood to be equivalent to a formula:

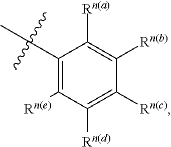

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance. Likewise, when a group R is defined as four substituents, R is understood to represent four independent substituents, R^{a}, R^{b}, R^{c}, and R^{d}. Unless indicated to the contrary, the substituents are not limited to any particular order or arrangement.

The present inventors have determined that HDAC inhibitors such as belinostat (also known as PXD-101) (N hydroxy-3-(3-phenylsulfamoyl-phenyl)-acrylamide) (and others), when given via these local administration routes, achieve very high intraocular concentrations, but of very brief duration. The present inventors have already determined that belinostat is effective for killing human retinoblastoma cells in vitro. The present inventors have additionally determined the pharmacokinetics of belinostat when injected directly into the vitreous of the rabbit eye and have calculated its intraocular half-life in vivo. The present inventors have determined that human retinoblastoma cells can be killed by exposure to a brief pulse of HDAC inhibitors, such as belinostat (and also etinostat, panobinostat, vorinostat, and romidepson), at multiple concentrations. For example, the present inventors have likewise determined the efficacy of belinostat at multiple concentrations, such as IC50 and IC90 for example, when these human retinoblastoma cells are exposed to belinostat for a time period equivalent to real-life exposure times in a rabbit model. Specifically, once the PK curve of intravitreally-injected belinostat was determined and the half-life calculated, human retinoblastoma cells were incubated in cell culture in media containing varying concentrations of belinostat for a length of time equivalent to 5 intraocular half-lives, the media was then replaced with belinostat-free media and incubated for 7 days, and the number of surviving cells was then determined. Then, as an example, the present inventors back-calculated the initial intravitreal injection dose that would ensure that a concentration equal to or above the in vitro IC90 would be achieved even out to 5 half-lives, in vivo. This method can be used to help determine suitable doses, effectiveness of serial injections, and other aspects of administration for compounds of the present invention.

HDAC inhibitors are currently given systemically, and systemically-administered drugs tend to not reach intraocular concentrations that are adequate to kill retinoblastoma cells. Additionally, HDAC inhibitors are currently given continuously over many days and for many cycles, in order to gradually effect change on cancer cell gene expression and regulation, and tend not to work when only given a single time. HDAC inhibitors are generally used for the treatment of hematologic malignancies, and have not been shown to work for solid tumors.

In contrast, the present inventors have discovered the use of HDAC inhibitors via the local delivery route (not systemic administration), to achieve much higher local concentrations than can be achieved via systemic administration, but for a much briefer exposure period (only a couple hours). The result of the present invention is superior and unexpected.

The present inventors believe that this combination of novel administration route and different pharmacokinetics can lead to the effective use of HDAC inhibitors and other drugs that alter histone modification (such as belinostat and others) for the treatment of a solid tumor, retinoblastoma.

### Compounds of the Present Invention

Examples of compounds of the present invention include those that inhibit HDAC (histone deacetylase) activity. One of ordinary skill in the art is readily able to determine whether or not a candidate is an HDAC inhibitor (HDACi). For example, assays which may conveniently be used to assess HDAC inhibition are described in Watkins et al., 2002, international (PCT) patent publication number WO 02/30879.

In other embodiments, the compounds of the present invention are certain active carbamic acid compounds which inhibit HDAC activity.

On example of the HDACi of the present invention are carbamic acid compounds comprising a sulfonamide linkage, as described, for example, in Watkins, C., et al., 2002, published international (PCT) patent application number WO 02/30879, incorporated herein by reference in its entirety. These compounds are also described in U.S. Pat. Nos. 6,888,027 and 8,835,501, both of which are incorporated herein by reference in its entirety.

One example of such a carbamic acid is a compound of the following formula:

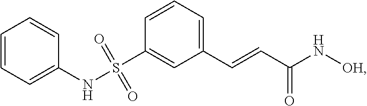 [SMILES: O=C(/C=C/c1cccc(S(=O)(=O)Nc2ccccc2)c1)NO]

known as belinostat, or PXD-101. Thus, PXD-101 is a compound of the present invention, and pharmaceutically acceptable salts, solvates, amides, esters, ethers, chemically protected forms, and prodrugs thereof.

In other embodiments, the compounds of the present invention are compound of the following formula:

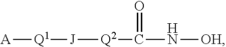

wherein:

A is a C₆₋₂₀carboaryl, or a C₅₋₂₀ heteroaryl group, and is substituted or unsubstituted;

Q¹ is a covalent bond, C₁₋₇alkylene, C₂₋₇alkenylene, and is unsubstituted or substituted;

J is

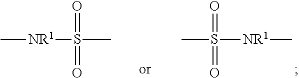

R¹ is H, C₁₋₇alkyl, C₃₋₂₀ heterocyclyl, C₆₋₂₀carboaryl, C₅₋₂₀ heteroaryl, C₆₋₂₀carboaryl-C₁₋₇alkyl, or C₅₋₂₀ heteroaryl-C₁₋₇alkyl, and is unsubstituted or substituted;

Q² is C₆₋₂₀carboarylene, C₅₋₂₀heteroarylene, C₆₋₂₀carboarylene-C₁₋₇alkylene, C₅₋₂₀heteroarylene-C₁₋₁₇alkylene, C₆₋₂₀carboarylene-C₂₋₇alkenylene, C₅₋₂₀heteroarylene-C₂₋₇alkenylene, C₁₋₇alkylene-C₆₋₂₀carboarylene, C₁₋₇alkylene-C₅₋₂₀heteroarylene, C₂₋₇alkenylene-C₈₋₂₀carboarylene, C₂₋₇alkenylene-C₅₋₂₀heteroarylene, C₁₋₇alkylene-C₆₋₂₀carboarylene-C₁₋₇alkylene, C₁₋₇alkylene-C₅₋₂₀heteroarylene-C₁₋₇alkylene, C₂₋₇alkenylene-C₆₋₂₀carboarylene-C₁₋₇alkylene, C₂₋₇alkenylene-C₅₋₂₀heteroarylene-C₁₋₇alkylene, C₁₋₇alkylene-C₆₋₂₀carboarylene-C₂₋₇alkenylene, C₁₋₇alkylene-C₅₋₂₀heteroarylene-C₂₋₇alkenylene, C₂₋₇alkenylene-C₆₋₂₀carboarylene-C₂₋₇alkenylene, or C₂₋₇alkenylene-C₅₋₂₀heteroarylene-C₂₋₇alkenylene, and is unsubstituted or substituted; and pharmaceutically acceptable salts thereof.

In one embodiment, Q¹ is a covalent bond, J is —NR¹—SO₂—, and the compounds have the following formula:

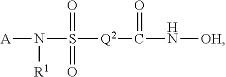

wherein the variables are defined above.

Group A:

In one embodiment, A is independently C₆₋₁₀carboaryl or C₅₋₁₀heteroaryl, and is unsubstituted or substituted (R^{A}).

In one embodiment, A is independently C₆carboaryl or C₅₋₆heteroaryl, and is unsubstituted or substituted.

In one embodiment, A is independently derived from: benzene, naphthalene, carbazole, pyridine, pyrrole, furan, thiophene, or thiazole; and is unsubstituted or substituted.

The phrase “derived from,” as used in this context, pertains to compounds which have the same ring atoms, and in the same orientation/configuration, as the parent cyclic group, and so include, for example, hydrogenated (e.g., partially saturated, fully saturated), carbonyl-substituted, and other substituted derivatives. For example, “pyrrolidone” and “N-methylpyrrole” are both derived from “pyrrole”.

In one embodiment, A is independently: phenyl, naphthyl, carbazolyl, pyridinyl, pyrrolyl, furanyl, thienyl, or thiazolyl; and is unsubstituted or substituted.

In one embodiment, A is independently phenyl, and is unsubstituted or substituted (e.g., with 1, 2, 3, 4, or 5 substituents).

In one embodiment, A is independently:

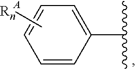

wherein n is 0, 1, 2, 3, 4, or 5.

If the substituent R^{A} is present, it is independent if more than one is present, and defined below.

In one embodiment, A is unsubstituted.

Thus, in a preferred embodiment, A is an optionally substituted phenyl group, Q¹ is a covalent bond, the compounds have the following formula:

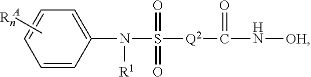

wherein the variables are defined above.

Group Q¹

The group Q¹ is independently a covalent bond, C₁₋₇alkylene, or C₂₋₇alkenylene, and is unsubstituted or substituted.

In one embodiment, Q¹ is independently a covalent bond or C₁₋₇alkylene, and is unsubstituted or substituted.

The term “alkylene,” as used herein, pertains to bidentate moieties obtained by removing two hydrogen atoms, either both from the same carbon atom, or one from each of two different carbon atoms, of a saturated hydrocarbon compound (a compound consisting of carbon atoms and hydrogen atoms) having from 1 to 20 carbon atoms (unless otherwise specified), which may be aliphatic (i.e., linear or branched) or alicyclic (i.e., cyclic but not aromatic).

The term “alkenylene,” as used herein, pertains to bidentate moieties obtained by removing two hydrogen atoms, either both from the same carbon atom, or one from each of two different carbon atoms, of a hydrocarbon compound (a compound consisting of carbon atoms and hydrogen atoms) having from 1 to 20 carbon atoms (unless otherwise specified) and having at least one carbon-carbon double bond, and which may be aliphatic (i.e., linear or branched) or alicyclic (i.e., cyclic but not aromatic).

In a preferred embodiment, Q¹ is a covalent bond.

Group J:

J is

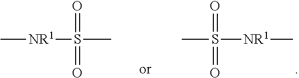

Group R¹:

The group R¹ is independently: —H, C₁₋₇alkyl, C₃₋₂₀heterocyclyl, C₆₋₂₀carboaryl, C₅₋₂₀heteroaryl, C₆₋₂₀carboaryl-C₁₋₇alkyl, or C₅₋₂₀heteroaryl-C₁₋₇alkyl, and is unsubstituted or substituted.

In one embodiment, R¹ is independently: —H or C₁₋₇alkyl, and is unsubstituted or substituted.

In one embodiment, R¹ is independently: —H or unsubstituted C₁₋₄alkyl.

In one embodiment, R¹ is independently: —H or unsubstituted saturated C₁₋₄alkyl.

In one embodiment, R¹ is independently: —H or unsubstituted saturated aliphatic C₁₋₄alkyl.

In one embodiment, R¹ is independently: —H, -Me, -Et, -nPr, -iPr, -nBu, -sBu, -iBu, or -tBu.

In one embodiment, R¹ is independently: —H, -Me, or -Et.

In one embodiment, R¹ is independently: —H or -Me.

The Q¹ Group:

In some embodiments, Q² is C₆₋₂₀carboarylene, C₅₋₂₀heteroarylene, C₆₋₂₀carboarylene-C₁₋₇alkylene, C₅₋₂₀heteroarylene-C₁₋₁₇alkylene, C₆₋₂₀carboarylene-C₂₋₇alkenylene, C₅₋₂₀heteroarylene-C₂₋₇alkenylene, C₁₋₇alkylene-C₆₋₂₀carboarylene, C₁₋₇alkylene-C₅₋₂₀heteroarylene, C₂₋₇alkenylene-C₈₋₂₀carboarylene, C₂₋₇alkenylene-C₅₋₂₀heteroarylene, C₁₋₇alkylene-C₆₋₂₀carboarylene-C₁₋₇alkylene, C₁₋₇alkylene-C₅₋₂₀heteroarylene-C₁₋₇alkylene, C₂₋₇alkenylene-C₆₋₂₀carboarylene-C₁₋₇alkylene, C₂₋₇alkenylene-C₅₋₂₀heteroarylene-C₁₋₇alkylene, C₁₋₇alkylene-C₆₋₂₀carboarylene-C₂₋₇alkenylene, C₁₋₇alkylene-C₅₋₂₀heteroarylene-C₂₋₇alkenylene, C₂₋₇alkenylene-C₆₋₂₀carboarylene-C₂₋₇alkenylene, or C₂₋₇alkenylene-C₅₋₂₀heteroarylene-C₂₋₇alkenylene, and is unsubstituted or substituted; and pharmaceutically acceptable salts thereof.

In one embodiment, Q² is independently: C₆₋₂₀carboarylene-C₂₋₇alkenylene, phenylene, phenylene-C₁₋₇alkylene, or phenylene-C₂₋₇alkenylene, and is unsubstituted or substituted.

In one embodiment, Q² is:

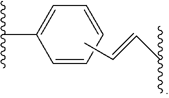

In one embodiment, Q² is:

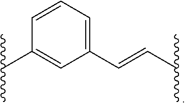 [SMILES: */C=C/c1cccc(*)c1]

The R^{A} substituent:

R^{A}, if present, is substituted or unsubstituted and independently selected from: C₁₋₇ alkyl, C₃₋₂₀ heterocyclyl, C₆₋₂₀ carboaryl, C₅₋₂₀ heteroaryl, C₆₋₂₀ carboaryl-C₁₋₇alkyl, or C₅₋₂₀ heteroaryl-C₁₋₇alkyl, carboxylic acid; ester; amido or thioamido; acyl; halo; cyano; nitro; hydroxy; ether; thiol; thioether; acyloxy; carbamate; amino; acylamino or thioacylamino; aminoacylamino or aminothioacylamino; sulfonamino; sulfonyl; sulfonate; sulfonamido; oxo; imino; hydroxyimino; C₅₋₂₀aryl-C₁₋₇alkyl; C₅₋₂₀aryl; C₃₋₂₀heterocyclyl; C₁₋₇alkyl; and bi-dentate di-oxy groups.

In one preferred embodiment, the HDAC inhibitor is selected from compounds of the following formula:

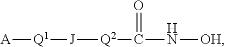

wherein:

A is independently: phenyl, and is unsubstituted or substituted;

Q¹ is independently: a covalent bond, —CH₂—, —CH₂—CH₂—, —CH₂═CH₂—;

J is

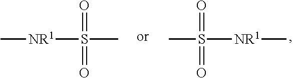

wherein

R¹ is H, C₁₋₇ alkyl, and is unsubstituted;

Q² is independently: phenylene-C₁₋₄alkylene, phenylene-C₂₋₄alkenylene, and is unsubstituted; and pharmaceutically acceptable salts, solvates, amides, esters, ethers, chemically protected forms, and prodrugs thereof.

In another embodiment, the HDAC inhibitor of the present invention is a compound of the following

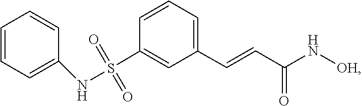 [SMILES: O=C(/C=C/c1cccc(S(=O)(=O)Nc2ccccc2)c1)NO]

and pharmaceutically acceptable salts, solvates, amides, esters, ethers, chemically protected forms, and prodrugs thereof.

In other embodiments of the present invention, the compound is Entinostat, also known as SNDX-275 and MS-275.

In these embodiments the compound is of the following formula:

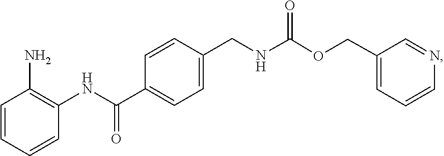 [SMILES: Nc1ccccc1NC(=O)c1ccc(CNC(=O)OCc2cccnc2)cc1]

and pharmaceutically acceptable salts, solvates, amides, esters, ethers, chemically protected forms, and prodrugs thereof.

In another embodiment of the present invention, the compound is panobinostat, also known as LBH-589.

In these embodiments, the compound is of the following formula:

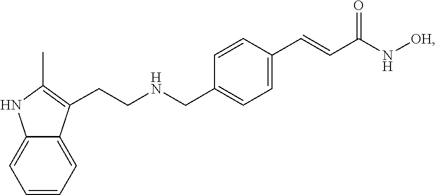 [SMILES: Cc1[nH]c2ccccc2c1CCNCc1ccc(/C=C/C(=O)NO)cc1]

and pharmaceutically acceptable salts, solvates, amides, esters, ethers, chemically protected forms, and prodrugs thereof.

In other embodiments, the compound is vorinostat, also known as suberanilohydroxamic acid (SAHA).

In these embodiments, the compound is of the following formula:

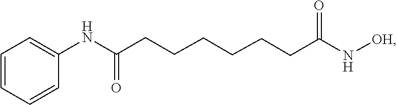 [SMILES: O=C(CCCCCCC(=O)Nc1ccccc1)NO]

and pharmaceutically acceptable salts, solvates, amides, esters, ethers, chemically protected forms, and prodrugs thereof.

In other embodiments, the compound is romidepsin, also known as depsipeptide or FK228.

In these embodiments, the compound is of the following formula:

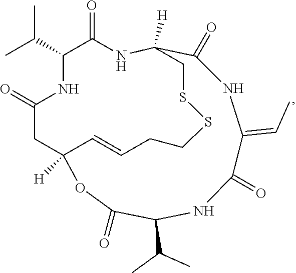 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

and pharmaceutically acceptable salts, solvates, amides, esters, ethers, chemically protected forms, and prodrugs thereof.

In another embodiment of the present invention, the HDAC inhibitor can be any molecule that inhibits the biological activity of a mammalian histone deacetylase (HDAC), including a human HDAC. Examples of useful HDAC inhibitors include, but are not limited to, suberoylanilide hydroxamic acid (SAHA), Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives or modifications or physiologically acceptable salt(s) of any HDACi molecule, that retain HDAC-inhibitory biological activity.

The term “derivative,” refers to HDAC inhibitors that are modified by covalent conjugation to other therapeutic or diagnostic agents or moieties, or to a label or marker (e.g., a radionuclide or one or more various enzymes), or are covalently conjugated to a protein, such as an immunoglobulin Fc domain or other “carrier” molecule, or to a polymer, such as polyethylene glycol (PEGylation) or biotin (biotinylation).

The term “carbo,” “carbyl,” “hydrocarbon” and “hydrocarbyl,” as used herein, pertain to compounds and/or groups which have only carbon and hydrogen atoms.

The term “hetero,” as used herein, pertains to compounds and/or groups which have at least one heteroatom, for example, multivalent heteroatoms (which are also suitable as ring heteroatoms) such as boron, silicon, nitrogen, phosphorus, oxygen, and sulfur, and monovalent heteroatoms, such as fluorine, chlorine, bromine, and iodine.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “ring,” as used herein, pertains to a closed ring of from 3 to 10 covalently linked atoms, more preferably 3 to 8 covalently linked atoms.

The term “aromatic ring,” as used herein, pertains to a closed ring of from 3 to 10 covalently linked atoms, more preferably 5 to 8 covalently linked atoms, which ring is aromatic.

The term “heterocyclic ring,” as used herein, pertains to a closed ring of from 3 to 10 covalently linked atoms, more preferably 3 to 8 covalently linked atoms, wherein at least one of the ring atoms is a multivalent ring heteroatom, for example, nitrogen, phosphorus, silicon, oxygen, and sulfur, though more commonly nitrogen, oxygen, and sulfur.

The term “alicyclic,” as used herein, pertains to compounds and/or groups which have one ring, or two or more rings (e.g., spiro, fused, bridged), wherein said ring(s) are not aromatic.

The term “aromatic,” as used herein, pertains to compounds and/or groups which have one ring, or two or more rings (e.g., fused), wherein at least one of said ring(s) is aromatic.

The term “heterocyclic,” as used herein, pertains to cyclic compounds and/or groups which have one heterocyclic ring, or two or more heterocyclic rings (e.g., spiro, fused, bridged), wherein said ring(s) may be alicyclic or aromatic.

The term “heteroaromatic,” as used herein, pertains to cyclic compounds and/or groups which have one heterocyclic ring, or two or more heterocyclic rings (e.g., fused), wherein said ring(s) is aromatic.

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions especially include those suitable for delivery to the eye. The pharmaceutical compositions can be conveniently presented in unit dosage form and prepared by any of the methods well known in the art of pharmacy.

As described herein, delivery may be intravitreal administration. Additionally, Delivery may be by intra-arterial administration. Intra-arterial administration includes, but is not limited to, administration by an endovascular microcatheter, or via an intra-arterial intravascular sustained delivery device (or controlled delivery device). Controlled release formulations can be prepared by incorporating the HDAC inhibitor into an inert matrix that permits release by wither diffusion or leaching mechanisms. Slowly degenerating matrices can also be incorporated into the formulation, e.g., alginates, polysaccharides. Surface placement of a device on the surface of the eye is also contemplated. For example, on the conjunctiva, subconjunctival, or episcleral, scleral, for sustained release.

Aspects of the invention also include compositions comprising a HDACi and a pharmaceutically acceptable carrier suitable for ophthalmic administration, e.g., suitable for subconjunctival, intravitreal, or topical administration, e.g., using eye drops and the like. Such pharmaceutical compositions can be configured for administration to a patient by a wide variety of delivery ophthalmic routes, e.g., subconjunctival injection, or other ocular delivery routes and/or forms of administration known in the art. The inventive pharmaceutical compositions may be prepared in liquid form, e.g., for administration via eye drops, or may be in dried powder form, such as lyophilized form.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

The present invention provides uses for active compounds which are capable of inhibiting HDAC (for example, inhibiting HDAC activity, inhibiting formation of HDAC complexes, inhibiting activity of HDAC complexes), as well as methods of inhibiting HDAC activity, comprising contacting a cell with an effective amount of an active compound. In preferred embodiments, delivery of the compound is to the eye.

The term “active,” as used herein, pertains to compounds which are capable of inhibiting HDAC activity, and specifically includes both compounds with intrinsic activity (drugs) as well as prodrugs of such compounds, which prodrugs may themselves exhibit little or no intrinsic activity.

One of ordinary skill in the art is readily able to determine whether or not a candidate compound is active, that is, capable of inhibiting HDAC activity. For example, assays which may conveniently be used to assess the inhibition offered by a particular compound are described in U.S. Pat. No. 6,888,027.

In one aspect, the present invention provides antiproliferative agents delivered to the eye. The term “antiproliferative agent” as used herein, pertains to a compound which treats a proliferative condition (i.e., a compound which is useful in the treatment of a proliferative condition).

The terms “cell proliferation,” “proliferative condition,” “proliferative disorder,” and “proliferative disease,” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers. In a preferred embodiment, the proliferative condition is cancer in the eye, and retinoblastoma.

Thus, the invention provides active compounds for use in a method of treatment of the human or animal eye. Such a method may comprise administering to such a subject a therapeutically-effective amount of an active compound, preferably in the form of a pharmaceutical composition.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes at least one of a reduction in the rate of progress, a halt in the rate of progress, amelioration of the condition, and cure of the condition. Treatment as a prophylactic measure is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy. A preferred embodiment of a combination treatment and therapy of the present invention is a method of the present invention in combination with a method known to treat tumors of the eye, particularly retinoblastoma.

The subject (patient) may be an animal, especially a mammal. Preferably, the subject is a human.

### Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

The present inventors have demonstrated that the present invention leads to at least equivalent excellent efficacy to currently-used traditional chemotherapy agents, but with far superior toxicity profiles. To wit, in an animal model that the present inventors have developed of advanced intraocular retinoblastoma with difficult-to-treat vitreous seeds, the present inventors have shown that intravitreal belinostat (an HDAC inhibitor of the present invention), was found to be equally effective to standard-of-care intravitreal melphalan. Belinostat caused a 95% reduction in tumor burden compared to saline, while melphalan caused a 93% reduction in tumor burden (statistically equivalent). At the same time, intravitreal injections of current standard of care melphalan led to 66-90% reduction in measured retinal function (electroretinography amplitudes) as well as retinal structural damage, while intravitreal injection of the HDAC belinostat led to no such retinal toxicity or structural damage. Thus, the present inventors demonstrate that targeted inhibition of histone modification via a local delivery route leads to excellent efficacy without the toxicity seen with current standard-of-care treatments.

Regarding the delivery of HDAC inhibitors via other local delivery routes, the present inventors developed a small animal (rabbit) model of intra-arterial chemotherapy (IAC). The present inventors subsequently described an extensive toxicity assessment platform for this model. Used together with the above-described rabbit retinoblastoma xenograft model that develops retinal tumors and vitreous seeds, they have assessed HDAC inhibitors via this route as well. The purposed of this example is to assess the efficacy and toxicity of various traditional chemotherapies and molecularly-targeted antineoplastic agents by the intra-arterial route, as well as the intravitreal route discussed in the previous paragraph.

For the first small animal IAC model developed by the inventors and referenced above, the rabbit's dominant ophthalmic artery was endovascularly cannulated, and each drug was injected at various doses. For intravitreal experiments, 1-3 weekly injections of each drug were performed. For the assessment of ocular toxicity described above, the testing platform the inventors developed included a broad array of tests including assessment of retinal structure and function by electroretinography, photography, fluorescein angiography, OCT, and OCT-Angiography, both before and after treatment. Human WERI-Rb1 retinoblastoma xenografts were treated with either intravenous, intra-arterial, or intravitreal chemotherapy. Assessment of efficacy against vitreous seeds was by both direct quantification of seed burden (vitreous harvesting and cell counting) and by assessment of apoptosis induction by immunohistochemistry.

To determine efficacy and toxicity of intravitreal HDAC inhibitors for retinoblastoma treatment, vitreous seeds were generated in cyclosporine-immunosuppressed rabbits. 1,000,000 human WERI-Rb1 human retinoblastoma cells were injected into each eye. The seeds were allowed to grow for two weeks to form the types of “spherules” seen clinically with vitreous seeds. 350 μg belinostat injections were used (equivalent to approximately 700 μg in a human-sized eye). The right eye was injected with belinostat three times, weekly. The left eye was injected with saline three times, weekly. The eyes were harvested for analysis two weeks after the final injection, and subjected to the cell quantification and apoptosis induction assays described in the above paragraph.

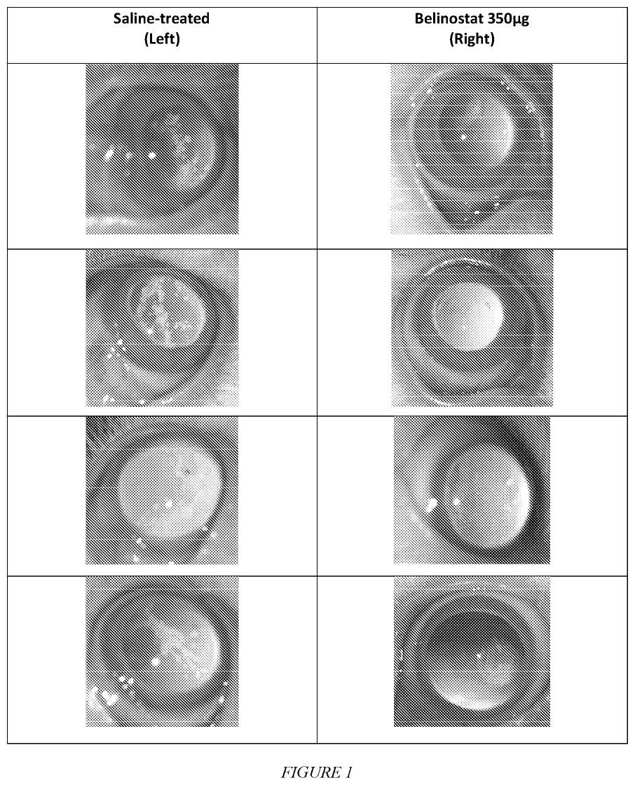

FIG. 1 contains a panel of photographs showing response to treatment of multiple rabbits' eyes treated with human WERI-Rb1 cells. The first column is all the left eyes treated with injections of saline (control), and the second column is all the right eyes treated with belinostat in accordance with an aspect of the present invention. There remains a massive disease burden of vitreous tumor seeds in the saline-treated left eyes, while belinostat resulted in eradication of retinoblastoma vitreous seeds.

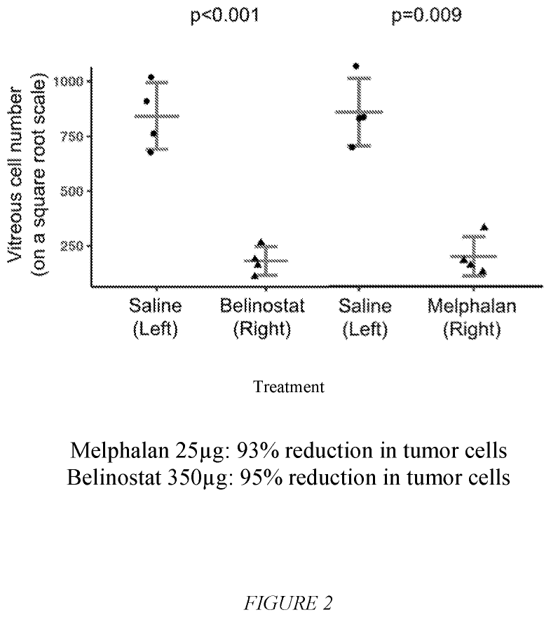

To compare the efficacy of an embodiment of the present invention with melphalan treatment, rabbit eyes were treated with 3 weekly injections of 350 μg belinostat by local (intravitreal) injection as described above. The melphalan comparator group received 3 weekly injections of 25 μg melphalan (the clinically-used dose) by intravitreal injection. Eyes were harvested two weeks after the final injection. FIG. 2 is a graph showing efficacy of melphalan (current standard-of-care)-treated eyes and belinostat treated eyes in accordance with an aspect of the present invention. Each drug was delivered by the local delivery route (intravitreal injection) as described above. The melphalan-treated eyes showed a 93% reduction in tumor cells (p=0.009) and the belinostat treated eyes showed a 95% reduction in tumor cells (p<0.001). The 93% reduction with melphalan and the 95% reduction with belinostat were statistically equivalent. The data in FIG. 2 show that belinostat treatment in accordance with the present invention is as effective as a standard of care melphalan treatment, eradicating essentially all tumor cells with only 3 injections given over less than one month.

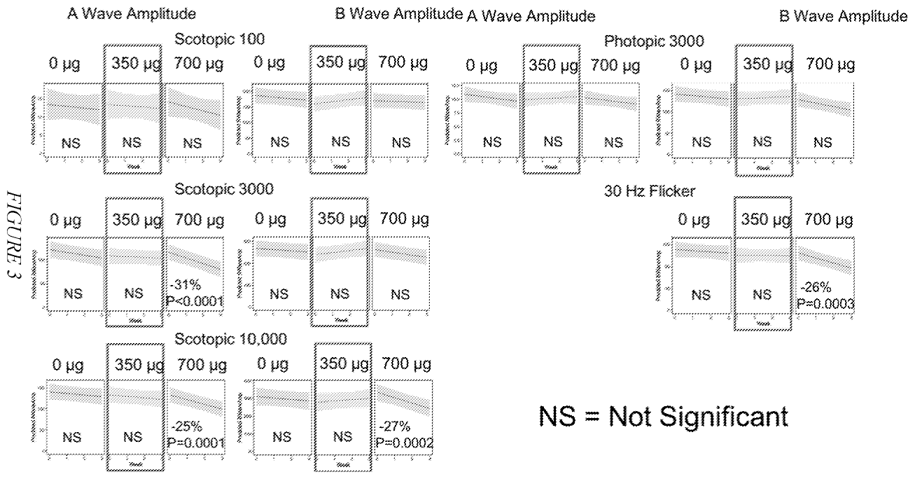

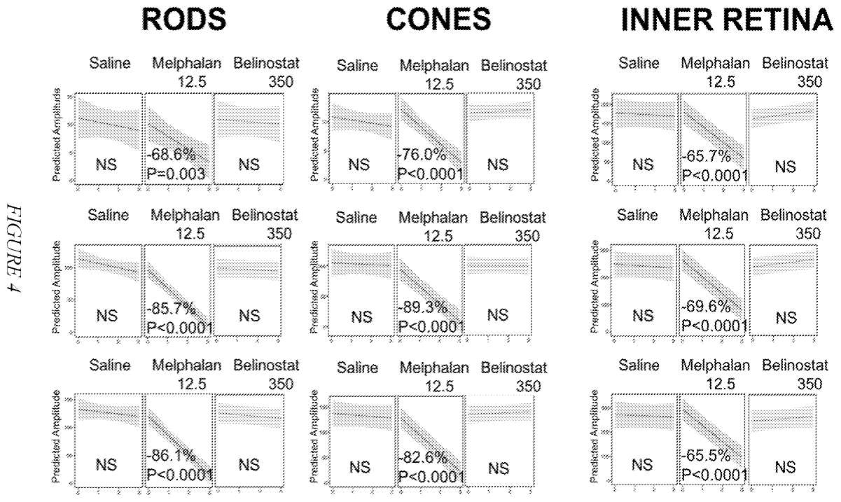

We next wanted to use the toxicity assessment platform that we have developed previously (described above) to compare the toxicity of belinostat (in accordance with the present invention) to current standard-of-care melphalan. As with the efficacy experiments, rabbits received either 3 injections (weekly) of belinostat 350 μg (the effective dose in our rabbit model), or else belinostat 700 μg (double the clinically-effective dose in our rabbit model), or else melphalan 25 μg (the current, clinically-used dose). FIGS. 3 and 4 demonstrate the measures of retinal function as measured by electroretinography (the standard measure of global retinal function in the field). FIG. 3 shows the various parameters (measures) of retinal function for control eyes receiving saline, for eyes receiving 3 injections of intravitreal belinostat 350 μg, and for eyes receiving 3 injections of intravitreal belinostat 700 μg. In the saline control group, and in the group receiving belinostat at the clinically-effective dose of 350 μg (see above), there was no worsening of ERG parameters, indicating no worsening of retinal function. Even in the group receiving double the clinically-effective dose (700 μg) of belinostat, there was only mild worsening of ERG parameters.

In FIG. 4, the absence of toxicity in the rabbits treated with the clinically-effective intravitreal dose of belinostat (350 μg) is compared to the excessive toxicity measured in eyes receiving current standard-of-care intravitreal melphalan dose of 25 μg. Massive retinal toxicity and huge reductions in retinal function are seen with standard-of-care melphalan injections, with reductions between 66-90% across all parameters measured. Thus, we demonstrate that intravitreal belinostat (in accordance with the present invention) does not cause retinal toxicity, as is otherwise seen with current standard-of-care treatments. Thus, we demonstrate that local delivery of an HDAC inhibitor, in accordance with the present invention, results in equal excellent efficacy compared to current standard-of-care treatment, but without the toxicities seen with current therapies.

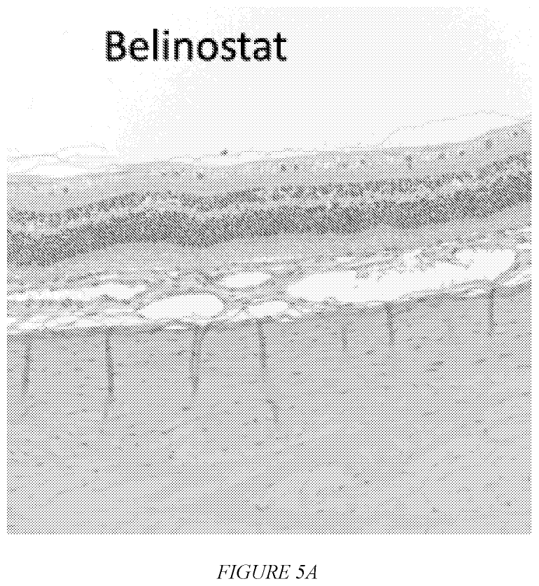

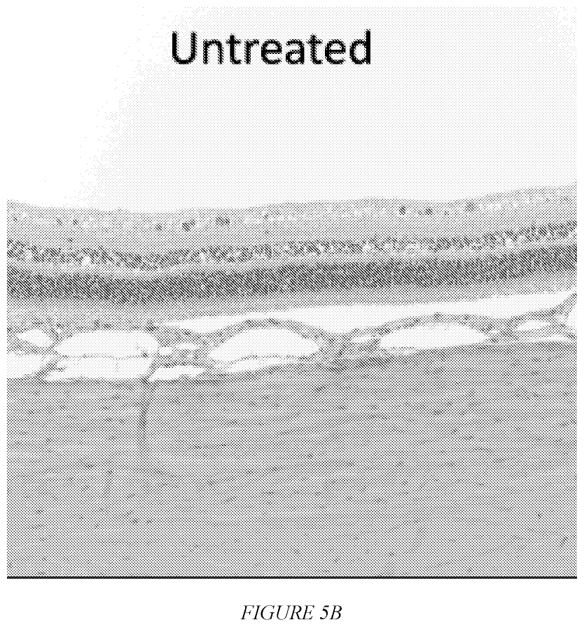

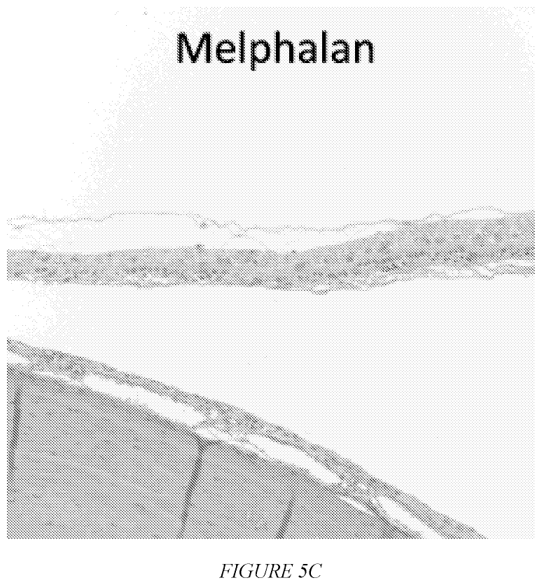

FIG. 5 shows the retained normal retinal architecture and healthy-appearing retina on histopathology following 3 weekly injections of belinostat, at a dose of either 350 μg or else at a dose of 700 μg (700 μg dose-treated eye is shown in the figure). The retina looks just as healthy as the saline-treated control eye. In contrast, the retina of an eye treated with typical 25 μg dose of standard-of-care intravitreal melphalan shows severe atrophy of the retina. This demonstrates that the present invention is significantly better in terms of reduced ocular toxicity and preserved retinal structure and function than currently-used treatments.

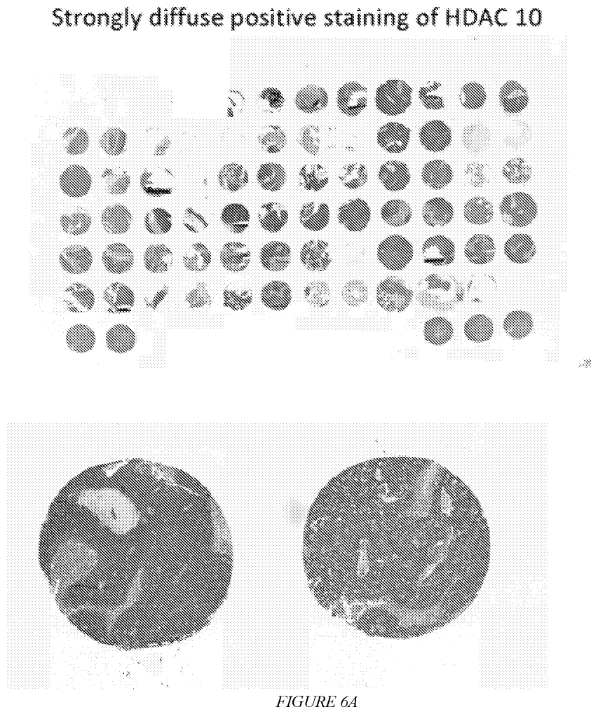

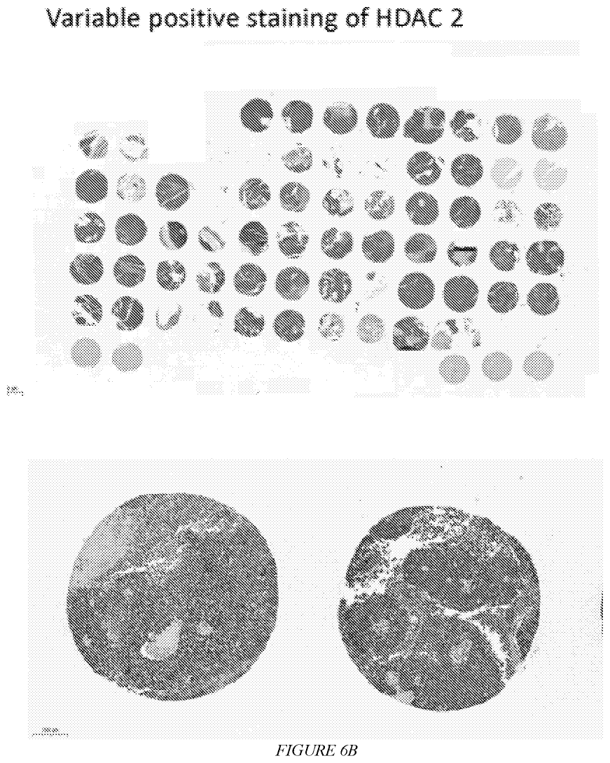

An important aspect of the present invention is that it does not assume that all HDACs have to be inhibited. In fact, we have performed analyses of dozens of human retinoblastoma tumors, and have determined that some HDACs are expressed in all (or almost all) patient tumors, while other are only expressed in the specific tumors of certain patients. We have determined the prevalence of expression of each HDAC across human retinoblastoma tumors from dozens of patients. FIG. 6A shows an example of a particular HDAC that is expressed ubiquitously across human retinoblastoma tumors, and in contrast, FIG. 6B shows an example of a particular HDAC that is only expressed in a few tumors. It should be obvious to those skilled in the art that only those HDACs expressed in a given tumor would need to be inhibited to achieve a clinical response, according to the present invention, and so the present invention includes both HDAC inhibitors that have a broad class effect, as well as those that only target a subset (or even a single) of HDACs.

The invention thus being described, it will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only.

1. A method of treating a proliferative condition of the eye, comprising:
administering to a patient a therapeutically effective amount of a composition that comprises a compound that inhibits the biological activity of a mammalian histone deacetylase (HDAC), or a pharmaceutically acceptable salt thereof; and a pharmaceutical carrier.

2. The method of claim 1, wherein the HDAC inhibiting compound is suberoylanilide hydroxamic acid (SAHA), Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (TM-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate).

3. The method of claim 1, wherein the HDAC inhibiting compound is Entinostat, Panobinostat, Belinostat, or Romidepsin.

4. The method of claim 1, wherein the HDAC inhibitor is a compound of the following formula: 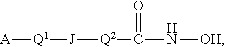
wherein:
A is independently: phenyl, and is unsubstituted or substituted;
Q¹ is independently: a covalent bond, —CH₂—, —CH₂—CH₂—, —CH₂═CH₂—;
J is 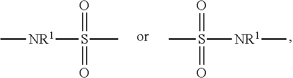
wherein
R¹ is H, C₁₋₇ alkyl, and is unsubstituted;
Q² is independently: phenylene-C₁₋₄alkylene, phenylene-C₂₋₄alkenylene, and is unsubstituted; and pharmaceutically acceptable salts, solvates, amides, esters, ethers, chemically protected forms, and prodrugs thereof.

5. The method of claim 1, wherein the HDAC inhibitor of the present invention is a compound of the following 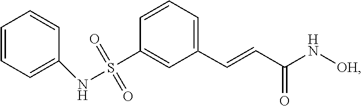 [SMILES: O=C(/C=C/c1cccc(S(=O)(=O)Nc2ccccc2)c1)NO]
and pharmaceutically acceptable salts, solvates, amides, esters, ethers, chemically protected forms, and prodrugs thereof.

6. The method of claim 1, wherein the HDAC inhibitor is a compound of the following formula: 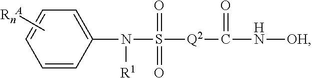
wherein:
R₁ is H, C₁₋₇ alkyl, C₃₋₂₀ heterocyclyl, C₆₋₂₀ carboaryl, C₅₋₂₀ heteroaryl, C₆₋₂₀ carboaryl-C₁₋₇alkyl, or C₅₋₂₀ heteroaryl-C₁₋₇alkyl, and is unsubstituted or substituted;
Q² is C₆₋₂₀carboarylene-C₂₋₇alkenylene, phenylene, phenylene-C₁₋₇alkylene, or phenylene-C₂₋₇alkenylene, and is unsubstituted or substituted;
R^{A} is, if present, is substituted or unsubstituted and independently selected from: C₁₋₇ alkyl, C₃₋₂₀ heterocyclyl, C₆₋₂₀ carboaryl, C₅₋₂₀ heteroaryl, C₆₋₂₀ carboaryl-C₁₋₇alkyl, or C₅₋₂₀ heteroaryl-C₁₋₇alkyl, carboxylic acid; ester; amido or thioamido; acyl; halo; cyano; nitro; hydroxy; ether; thiol; thioether; acyloxy; carbamate; amino; acylamino or thioacylamino; aminoacylamino or aminothioacylamino; sulfonamino; sulfonyl; sulfonate; sulfonamido; oxo; imino; hydroxyimino; C₅₋₂₀aryl-C₁₋₇alkyl; C₅₋₂₀aryl; C₃₋₂₀heterocyclyl; C₁₋₇alkyl; and bi-dentate di-oxy groups; and
n is 0, 1, 2, 3, 4, or 5;
and pharmaceutically acceptable salts, solvates, amides, esters, ethers, chemically protected forms, and prodrugs thereof.

7. The method of claim 1, wherein the cancer is ocular tumors.

8. The method of claim 1, wherein the cancer is retinoblastoma.

9. The method of claim 1, wherein the administration step further comprises intravitreal injection.

10. The method of claim 1, wherein the administration step further comprises intra-arterial local delivery of the HDAC inhibitor.

11-30. (canceled)

31. A method of treating retinoblastoma in a subject in need thereof, comprising:
administering to a patient a therapeutically effective amount of a compound of the following formula: 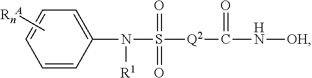
wherein:
R₁ is H, C₁₋₇ alkyl, C₃₋₂₀ heterocyclyl, C₆₋₂₀ carboaryl, C₅₋₂₀ heteroaryl, C₆₋₂₀ carboaryl-C₁₋₇alkyl, or C₅₋₂₀ heteroaryl-C₁₋₇alkyl, and is unsubstituted or substituted;
Q² is C₆₋₂₀carboarylene-C₂₋₇alkenylene, phenylene, phenylene-C₁₋₇alkylene, or phenylene-C₂₋₇alkenylene, and is unsubstituted or substituted;
R^{A} is, if present, is substituted or unsubstituted and independently selected from: C₁₋₇ alkyl, C₃₋₂₀ heterocyclyl, C₆₋₂₀ carboaryl, C₅₋₂₀ heteroaryl, C₆₋₂₀ carboaryl-C₁₋₇alkyl, or C₅₋₂₀ heteroaryl-C₁₋₇alkyl, carboxylic acid; ester; amido or thioamido; acyl; halo; cyano; nitro; hydroxy; ether; thiol;
thioether; acyloxy; carbamate; amino; acylamino or thioacylamino; aminoacylamino or aminothioacylamino; sulfonamino; sulfonyl; sulfonate; sulfonamido; oxo; imino; hydroxyimino; C₅₋₂₀aryl-C₁₋₇alkyl; C₅₋₂₀aryl; C₃₋₂₀heterocyclyl; C₁₋₇alkyl; and bi-dentate di-oxy groups; and
n is 0, 1, 2, 3, 4, or 5;
and pharmaceutically acceptable salts, solvates, amides, esters, ethers, chemically protected forms, and prodrugs thereof.

32. The method of claim 31, wherein the compound is of the following formula: 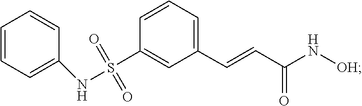 [SMILES: O=C(/C=C/c1cccc(S(=O)(=O)Nc2ccccc2)c1)NO]
and pharmaceutically acceptable salts, solvates, amides, esters, ethers, chemically protected forms, and prodrugs thereof.

33. The method of claim 31, further comprising the intravitreal injection administration of the compound.

34. The method of claim 31, further comprising the intra-arterial administration of the compound.

35. The method of claim 31, further comprising co-administration with a known anti-cancer agent.
